(19) United States
(12) Patent Application Publication
(10) Pub. No.: US 2023/0032992 A1
(43) Pub. Date: Feb. 2, 2023

(54) DEVICES, SYSTEMS, AND METHODS FOR PLASMA TREATMENT OF PRODUCTS IN AN EDIBLE COATING SYSTEM

(71) Applicant: Apeel Technology, Inc., Goleta, CA (US)

(72) Inventors: Krishnan Chari, Santa Barbara, CA (US); Anuj Purohit, Goleta, CA (US); Heather Omerigic, Goleta, CA (US); Taylor Person, Goleta, CA (US)

(21) Appl. No.: 17/815,356

(22) Filed: Jul. 27, 2022

Related U.S. Application Data
(60) Provisional application No. 63/226,621, filed on Jul. 28, 2021.

(51) Int. Cl. A23P 20/18; B05D 3/14; B05D 1/02

(52) U.S. Cl. CPC A23P 20/18; B05D 3/142; B05D 3/148; B05D 1/02

(57) ABSTRACT

Disclosed herein is a plasma coating system, including a plasma treatment device configured to generate a plasma discharge to treat a product; a coating station that treats the product with a liquid coating; and a transport surface that transports a product from the plasma treatment device to the coating station.

### CLAIM OF PRIORITY

This application claims priority under 35 USC § 119(e) to U.S. Patent Application Ser. No. 63/226,621, filed on Jul. 28, 2021, the entire contents of which are hereby incorporated by reference.

### TECHNICAL FIELD

This document describes devices, systems, and methods related to coating of products, such as devices, systems, and methods for plasma-treating products and applying a coating to plasma-treated products.

### BACKGROUND

Common products, such as food products, agricultural products, and fresh produce, are susceptible to degradation and decomposition (i.e., spoilage) when exposed to the environment. Product degradation can occur via abiotic means as a result of evaporative moisture loss from an external surface of the agricultural products to the atmosphere, oxidation by oxygen that diffuses into the agricultural products from the environment, mechanical damage to the surface, and/or light-induced degradation (i.e., photodegradation).

Many products are handled in packing houses, where they are sorted and packaged. On some commercial packing lines, agricultural products may be treated, for example, with waxes which preserve the agricultural products, with sanitizing agents which reduce or eliminate bacteria or other biotic stressors, and/or with solutions that can form protective coatings over the products.

### SUMMARY

Efficient application of protective coatings can enhance product shelf life and increase resistance to microbial load, spoilage, degradation, or mechanical damage while reducing resources used in coating products. Some embodiments described herein include devices, systems, and methods for plasma treatment of products, such as food products, non-food products, packages, produce, and other perishable or degradable products. Bulk plasma treatment of products includes generating an area of plasma discharge and disposing products therein (e.g., increasing product surface energy). The energized products are then provided to a coating station which applies a liquid coating material to the product. The increased surface energy increases wettability of the product surface in contact with the liquid coating material. In some embodiments, this increases the efficiency of forming a coating on product surfaces.

Various embodiments described herein include one or more mechanisms that expose a product surfaces to a plasma discharge and apply a liquid coating material to the exposed product. In some embodiments, a plasma treatment device generates an electric field on a discharge probe sufficient to partially ionize the surrounding gas and emit a plasma discharge. The plasma discharge shape depends on the shape of the discharge probe and additional discharge probes emit additional corresponding plasma discharges. A product is exposed to the plasma discharge for a time period, increasing the surface energy of the product surface, for example. In some embodiments, the increased product surface energy increases the product wettability.

In some embodiments, the discharge electrode of the plasma treatment device is arranged proximal to a product transport mechanism, such as a conveyor or brush bed. The plasma discharge emitted from the discharge electrode covers a surface area (e.g., a treatment area) of the product transport mechanism. The product transport mechanism transports the product through the stationary surface area thereby exposing the product to the plasma discharge within the treatment area. The product transport velocity can determine the treatment time within the plasma discharge. In some embodiments, the product transport mechanism induces multi-axis rotation (e.g., tumbling) in the product during transportation. The tumbling product can expose a large surface area (e.g., the complete surface area of the product) to the plasma discharge.

In some embodiments, the exposed product is optionally provided to a coating device that applies a liquid coating material to the product. In some embodiments, the liquid coating material is applied to the product, or to the product transport mechanism and thereby to the product, by one or more sprayers. In various additional embodiments, the product is submerged in liquid coating material by a submersion coating device. In some example embodiments, increased wettability of the product surfaces decreases the contact angle between the liquid coating material and product surfaces and facilitates more efficient coverage of the product surface area by the liquid coating material.

Some embodiments of the devices, systems, and techniques described herein may provide one or more of the following advantages. First, some embodiments described herein increase the coating efficiency of the coating device. By increasing the surface energy of the product surface, coating material applied to the product surface can experience a lower contact angle and therefore a particular volume of coating material can cover more surface area than a product with a lower surface energy. The overall amount of coating material to envelope a product can be reduced while achieving a predictable coating thickness and promoting consistency across the product surface.

Second, various example embodiments (e.g., utilizing non-thermal plasma discharge) increase the surface energy of the product without inducing irreversible changes (e.g., damage) to the product. Non-thermal plasma discharges operate at a bulk temperature near room temperature (e.g., below 100° F.) for example, which can increase the surface energy and not significantly increase the surface temperature of the product.

Third, various example embodiments can reduce the microbial load present on product surfaces through deactivation. For example, reducing the microbial load on food products increases the shelf-stability of the product and reduces downstream bacterial outbreaks. In some embodiments, the coated product can be exposed to a second plasma discharge reducing the microbial load present in exposed coating material.

Fourth, various embodiments promote coating adherence, decreasing clean-in-place operations and/or handling of excessing coating material shed from products during/subsequent to the coating operation. For example, increased product surface energy can increase the adhesive force between the coating material and the product surface, decreasing the amount of excess coating material shed from the product surface in processes following the coating material application. In some embodiments, increased surface energy of products facilitates adherence of coating material such that which can be applied at lower rates and thereby reduce coating material usage, over-spray, etc.

Fifth, various embodiments described herein facilitate effective application of a coating material to product without the addition of wetting agents to the coating material or with a relatively low content of added wetting agents. The coating material can be formulated to provide desired barrier properties, stability, and other performance characteristics with less dependence on wetting characteristics.

Sixth, various embodiments described herein facilitate efficient product coating using less liquid coating material resulting in reduced material costs. Products being coated in less liquid coating material promotes the effective removal of the liquid coating material solvent (e.g., drying) reducing times and energy costs associated with drying.

Seventh, various embodiments facilitate reduced drying time by promoting a consistent layer of coverage over the product surface with relatively less liquid coating material. For example, a consistent coating layer can be performed with less overall moisture to be removed in order to create the dried coating layer.

As additional description to the examples described below, the present disclosure describes the following examples.

Example 1 is a plasma coating system, including a plasma treatment device configured to generate a plasma discharge to treat a product; a coating station that treats the product with a liquid coating; and a transport surface that transports a product from the plasma treatment device to the coating station.

Example 2 is the system of example 1, wherein the plasma treatment device can be arranged above the transport surface.

Example 3 is the system of example 1 or example 2, wherein the transport surface can include a brush-bed transport surface.

Example 4 is the system of any one of the preceding examples, further including a drying station that receives products from the coating station and facilitates drying of the liquid coating to form a dried coating layer on the product.

Example 5 is the system of example 4, wherein the plasma coating system can include a second plasma treatment device configured to generate a second plasma discharge arranged between the coating station and the drying station.

Example 6 is the system of any one of the preceding examples, wherein the coating station can include an applicator selected from the group consisting of a spray applicator, a brush-bed applicator, and a submersion applicator.

Example 7 is the system of any one of the preceding examples, wherein the plasma treatment device can be configured to generate the plasma discharge based on a peak electric potential in a range from 1 V to 200 kV.

Example 8 is the system of any one of the preceding examples, wherein the plasma discharge can be a non-thermal plasma discharge with a bulk temperature below 200° F.

Example 9 is the system of any one of the preceding examples, wherein the plasma discharge covers an area in a range from 0.01 m2 to 10 m2.

Example 10 is the system of any one of the preceding examples, wherein an area of the plasma discharge can be defined by an electron density of at least 2012 cm−3.

Example 11 is the system of any one of the preceding examples, wherein an area of the plasma discharge can be defined by an electron temperature of at least 5,000 K.

Example 12 is the system of any one of the preceding examples, wherein a minimum distance between the plasma treatment device and the transport surface can be in a range from 5 cm to 15 cm.

Example 13 is the system of any one of the preceding examples, wherein a minimum distance between the plasma treatment device and the product can be in a range from 5 cm to 15 cm.

Example 14 is the system of any one of the preceding examples, wherein the plasma discharge covers a surface dimension of the transport surface in a range from 0.1 m to 10 m.

Example 15 is the system of any one of the preceding examples, wherein the transport surface can be configured to transport the product at a rate such that the product can be within the plasma discharge for a residence time, wherein the residence time can be in a range from 1 s to 300 s.

Example 16 is the system of any one of the preceding examples, wherein the coating material can include a monoglyceride and fatty acid salt.

Example 17 is the system of any one of the preceding examples, wherein the fatty acid salt can include a C16 fatty acid salt and a C18 fatty acid salt.

Example 18 is the system of any one of the preceding examples, wherein the product can be a perishable product.

Example 19 is the system of any one of the preceding examples, wherein the product can be a non-edible product.

Example 20 is a method for plasma treating and coating product, including generating a plasma discharge; exposing an external surface of a product to the plasma discharge; and applying a liquid coating material to the external surface of the product.

Example 21 is the method of example 20, wherein the plasma discharge can be a non-thermal discharge.

Example 22 is the method of example 20 or example 21, wherein exposing the product to the plasma discharge can include exposing the product for a time duration in a range from 1 s to 10 m.

Example 23 is the method of any one of examples 20-22, wherein the plasma discharge can be a non-thermal plasma discharge with a bulk temperature below 200° F.

Example 24 is the method of any one of examples 20-23, including drying the liquid coating material on the product.

Example 25 is the method of any one of examples 20-24, including, after applying the liquid coating material, exposing the product to a second plasma discharge.

Example 26 is the method of any one of examples 20-25, wherein the coating material can include a monoglyceride and fatty acid salt.

Example 27 is the method of any one of examples 20-26, wherein the fatty acid salt can include a C16 fatty acid salt and a C18 fatty acid salt.

Example 28 is the method of any one of examples 20-27, wherein the products can be perishable products.

Example 29 is the method of any one of examples 20-28, wherein the products can be non-edible products.

Example 30 is a plasma coating system, including means for generating a plasma discharge to treat a product; means for applying a liquid coating to the product; and a transport surface that transports a product proximate the means for generating a plasma discharge.

Example 31 is the system of example 30, wherein a minimum distance between the means for generating a plasma discharge and the transport surface can be in a range from 5 cm to 15 cm.

The details of one or more implementations are set forth in the accompanying drawings and the description below. Other features and advantages will be apparent from the description and drawings, and from the claims.

### BRIEF DESCRIPTION OF THE DRAWINGS

FIG. 1 schematically illustrates an example coating system including a plasma treatment device.

FIG. 2A schematically illustrates the plasma treatment device of FIG. 1.

FIG. 2B schematically illustrates a top-down view of a plasma treatment area.

FIG. 3A schematically illustrates an example plasma treatment device including three discharge electrodes arranged above a product transport system.

FIGS. 3B-3D schematically illustrates the composite treatment areas of various arrangements of the three discharge electrodes of FIG. 3A.

FIG. 4A schematically illustrates an example plasma treatment device including a wire-shaped discharge electrode arranged above a product transport system.

FIG. 4B schematically illustrates the treatment area of discharge electrode of FIG. 4A.

FIG. 5A schematically illustrates the contact angle between liquid coating material and a low surface energy surface.

FIG. 5B schematically illustrates the contact angle between liquid coating material and a high surface energy surface.

FIG. 6 is a flow chart detailing the steps of the plasma coating process.

FIG. 7 is a bar chart depicting the average mass loss rate of the cherry tomato treatment groups.

FIG. 8 is a bar chart depicting the average mass loss factor of the cherry tomato treatment groups for FIG. 7.

FIG. 9 is a line graph comparing the percentage of average shrinkage of cherry tomato treatment groups.

FIG. 10 is a bar chart depicting the average mass loss factor of kumquat treatment groups.

FIG. 11 is a bar chart depicting the CFU of yeast present on the surface of kumquat treatment groups.

### DETAILED DESCRIPTION OF ILLUSTRATIVE EMBODIMENTS

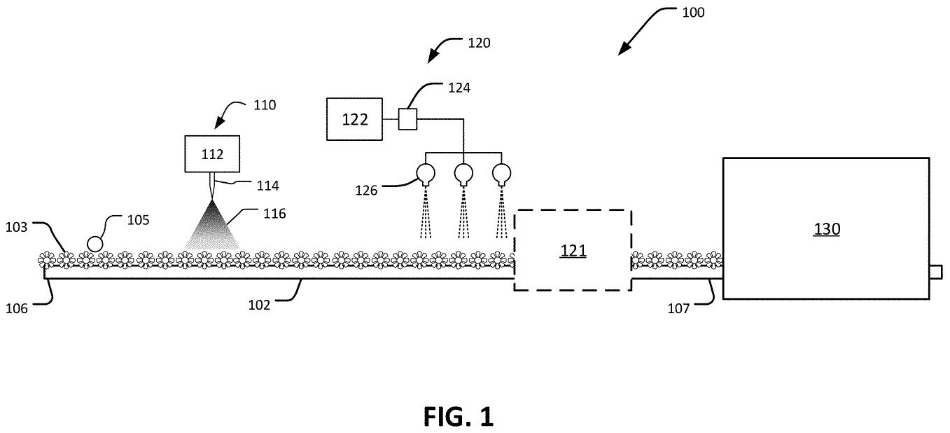

Referring to FIG. 1, an example coating system 100 is shown. The coating system 100 is configured to treat the surface of products, such as food products, harvested produce or other agricultural products, seeds, non-food products, packaging, etc., to form a coating on the products. The coating system 100 includes an apparatus to apply a plasma treatment to the surface of products, and/or to apply a coating material to the treated products. In some embodiments, the coating system 100 includes a drying tunnel 130 to facilitate drying of a coating solution (e.g., a high water-content coating).

The coating system 100 includes a transport surface (e.g., conveyor 102) that moves product through the treatment apparatuses of system 100. The conveyor 102 transports products between the respective treatment apparatuses of the system 100. For example, the conveyor 102 includes a conveyor bed that transports products from one end of the coating system 100 to the opposing end, such as via a rolling translating conveyer. In an example embodiment, the conveyor 102 of FIG. 1 is a continuous conveyor bed that extends from a first end 106 of the coating system 100 to a second, opposite end 107. Products, such as a product 105, move along conveyor 102 between the first and second ends. Alternatively or additionally, the conveyor 102 can be composed of multiple sequential conveyors arranged to coordinate the transport of product 105 through the coating system 100.

The conveyor 102 of FIG. 1 includes one or more brushes 103 arrayed along the longitudinal axis of the conveyor 102. The brushes 103 agitate the exposed surfaces of the product 105s (e.g., removing particulates). In an example embodiment, the brushes 103 are oriented such that distal ends contact the exposed product 105 surfaces. The conveyor 102 operates the brushes 103 to cause product 105 motion in parallel with the directionality of the conveyor 102, or cause product 105 tumbling motion to promote uniform and consistent exposure of product 105 surfaces to bristle agitation.

The coating system 100 includes a plasma treatment device 110 for plasma treating surfaces of the product 105. The plasma treatment device 110 includes a plasma apparatus 112 and a discharge electrode 114. The plasma treatment device 110 generates and emits a plasma discharge 116. In some embodiments, the plasma apparatus 112 supplies an electric potential to the discharge electrode 114. The electric potential generates an electric field of sufficient strength to ionize the gas molecules in the volume between the discharge electrode 114 and the conveyor 102. The ionized molecules and free electrons generate a plasma discharge 116 emitting from the discharge electrode 114 (e.g., nearest the conveyor 102 surface). In some embodiments, the discharge electrode 114 is within the plasma apparatus 112 and the plasma discharge 116 emits from the plasma apparatus 112.

The free electrons in the plasma discharge 116 create reactive species within the plasma discharge 116 such as reactive oxygen and reactive nitrogen species. When exposed to the plasma discharge 116 and reactive species therein, the surface chemistry of the product 105 is altered. In some embodiments, the surface charge is altered. For example, the plasma discharge 116 increases the surface energy of the product surface of product 105. In some embodiments, the product 105 surface energy is increased by 0.1 mN/m (mJ/m²) or more (e.g., 0.5 mN/m or more, 1 mN/m or more, or 5 mN/m or more). Increased product 105 surface energy increases the wettability of the product 105. In some embodiments, the introduction of the reactive oxygen species to the product 105 surface alters the surface chemistry by creating additional oxygenated bonds and increasing the surface hydrophilicity. Increased surface hydrophilicity increases the wettability of the product 105.

In some embodiments, coating system 100 includes a coating device 120 that can apply a coating material to products. For example, the coating device 120 includes a vessel 122 that defines a volume to hold and/or prepare liquid material and additives, a heating element 124, and one or more sprayers 126. The vessel 122 stores a coating mixture for application to product 105 surfaces traversing the conveyor 102 beneath. In some implementations, the coating device 120 includes one of the conveyors composing the multiple sequential conveyors of the coating system 100.

The vessel 122 dispenses coating mixture to the heating element 124 (e.g., an in-line heating element) which raises the temperature of the material to a predetermined temperature. Increasing the temperature of the coating mixture to a predetermined value, or maintaining the temperature at a predetermined value, can deactivate microbial load that may be present before/during application. In various example embodiments, the coating material is heated to a temperature between 50° C. and 100° C., 60° C. and 90° C., or about 80° C. In some examples, the temperature is between 55° C. and 65° C. just before exiting from a delivery nozzle (e.g., of a sprayer). The heated coating material is then directed to one or more sprayers 126 that disperse the liquid material onto exposed product 105 surfaces and/or brushes 103 of the brush bed conveyor 102.

In some embodiments, the coating device 120 includes a submersion coating device 121. For example, the product 105 is submerged in a vessel including a volume of coating material. Submersion in the vessel coats the external surface of the product 105 in the coating material, and can facilitate application of complex surfaces and/or of product 105 within a container.

Coating system 100 optionally includes drying tunnel 130. The drying tunnel 130 can include various components to facilitate drying (e.g., dehydrating) the coating mixture on the products, for example heated air blowers, drying brushes, and drying tunnels with roller conveyors. The drying tunnels may include air recirculation and/or humidity control systems that utilize ventilation ducts and modulating exhaust. High pressure blowers may be provided to supply air to a perforated plate, which can promote high air velocity across the product path.

In various example embodiments, the conveyor 102, plasma treatment device 110, and/or coating device 120, can be arranged before, within, and/or after drying tunnel 130. For example, treatment apparatus may include conveyor 102, plasma treatment device 110, and/or coating device 120 in sequence to treat product before the product enters drying tunnel 130. Alternatively or additionally, coating device and/or surface treatment device may be included at entry and/or exit locations of drying tunnel 130. In further example embodiments, conveyor 102, plasma treatment device 110, and/or coating device 120 can be arranged as described herein or in alternative sequences to provide increased removal of microbial load.

In some embodiments, the conveyor 102 and devices 110, 120, 121, and 130 can be used independently, in sequence, in parallel, or in any combination.

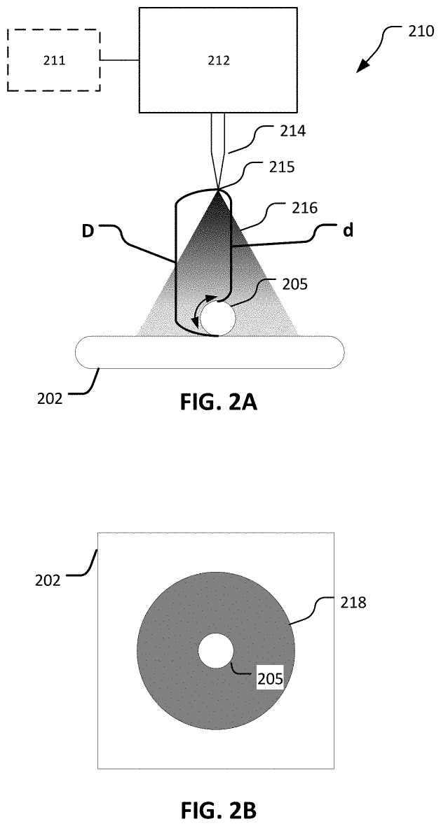

Referring to FIG. 2A, a plasma treatment device 210 for treating a product 205 is shown. The plasma treatment device 210 includes a plasma apparatus 212 which generates a potential at the discharge electrode 214. The plasma apparatus 212 includes a power supply, and/or an electrical connection for connecting plasma apparatus 212 to a power supply. In various example embodiments, plasma treatment device 210 includes one or more transformers, rectifiers, and/or control circuitry, to generate and sustain the potential at the discharge electrode. In some embodiments, the power supplied to the discharge electrode 214 is AC power, DC power, or RF power. In various example embodiments, plasma treatment device 210 includes one or more features described above with reference to plasma treatment device 110.

In some embodiments, the product 205 experiences linear motion along the neutral transport surface 202 during transportation and multi-axis rotational motion (e.g., tumbling, see arrows of FIG. 2) induced by the transport surface 202. For example, the brush bed conveyor 102 of FIG. 1 imparts a tumbling motion in a transported product 105 corresponding to the product 105 contact with brushes 103 undergoing rotational motion. Product 205 tumbling exposes larger portions of the product 205 surfaces to the plasma discharge 216, up to and including the entire product 205 surface. A vibratory mechanism which induces vibrations or tumbling in the product 205 can alternatively provide the product 205 tumbling motion.

Plasma treatment device 210 is arranged a distance from a transport surface 202 (e.g., an electrically neutral transport surface). For example, the plasma treatment device 210 is arranged such that a distance, D, separates the discharge electrode 214 from the electrically neutral transport surface 202. In some embodiments, the plasma treatment device 210 is capable of altering D, increasing or decreasing the separation. For example, the plasma treatment device 210 can alter D according to product size, product volume, desired treatment area 218 size, desired distance, d, between the discharge electrode and the product 205, or desired power density. In some embodiments, the plasma treatment device 210 alters D by moving the plasma apparatus 212, moving the discharge electrode 214, or controlling the transport surface 202 to move. The movement of the plasma treatment device 210 can be performed manually, e.g., by an operator, or automatically by the coating system 100 in response to a condition or command.

In some embodiments, the distance D between the discharge electrode 214 and the transport surface 202, and/or the distance d between the discharge electrode and the product 205, affects the treatment dosage. For example, the distances can be selected at least partially based on the operational power of the plasma apparatus 212, product 205 transport time within the plasma discharge 216 (e.g., residence time), and discharge electrode 214 shape. In various example embodiments distance D, and/or distance d, is in a range from 5 cm to 30 cm (e.g., 5 cm to 10 cm, 5 cm to 15 cm, 8 cm to 20 cm, 8 cm to 15 cm, or 20 cm to 30 cm). Larger distances can increase the treatment area 218 size or accommodate a large product 205, while smaller distances increase the power applied to the product 205 contact area and can reduce residence time. A consistent distance d increases treatment uniformity of products of differing sizes or contact areas.

In some embodiments, the plasma treatment device 210 includes a control panel 211 for manipulating the function of the plasma treatment device 210 by a user. For example, the control panel 211 can include one or more input and/or output devices, such as screens, displays, buttons, switches, dials, toggles, and/or interactive surfaces with which the user inputs information into the plasma treatment device 210 and/or receives output from the plasma treatment device 210. In various examples, the control panel 211 can be housed within a common housing with plasma treatment device 210, or housed externally and in communication with plasma treatment device 210.

For example, the user can input into the plasma treatment device 210 electric potential parameters, discharge electrode 214 number, discharge electrode 214 shape, distance, D, from the discharge electrode 214 to the transport surface, distance d from the discharge electrode 214 to the product surface, product 205 number, product 205 type, product 205 volume, and/or treatment time. In an example embodiment, the control panel includes an interface located on plasma treatment device 210. Alternatively or additionally, plasma treatment device 210 receives one or more control inputs from a remote source, such as a control interface associated with a treatment system that plasma treatment device 210 is a part of (e.g., via a wired or wired connection). In some embodiments, plasma treatment device 210 outputs data, such as an operational status of plasma treatment device 210, to the control interface.

The plasma apparatus 212 operates the discharge electrode 214 at an electric potential (e.g., voltage) to generate a plasma discharge 216 (e.g., a corona discharge) between the tip 215 of the discharge electrode 214 and an electrically grounded source, such as transport surface 202. In general, the composition of the gas surrounding the discharge electrode 214 affects the potential threshold to generate the plasma discharge 216. In some embodiments, the methods and systems described herein operate in the absence of purified feed gases. For example, the plasma apparatus 212 utilizes the ionization of atmospheric gases (e.g., at ambient temperatures and pressures) to generate the plasma discharge 216, and does not include a feed gas. In an example embodiment, the plasma treatment device 210 described herein is a corona discharge device producing a corona discharge. Alternatively or additionally, the plasma treatment device 210 includes a dielectric barrier discharge device, a capacitive discharge device, a gliding arc discharge device, or a plasma needle discharge device.

In various embodiments, the plasma discharge 216 generated (e.g., emitted) by the discharge electrode 214 is a non-thermal plasma (e.g., cold plasma). For example, non-thermal plasmas operate at a temperature of 200° F. or less (e.g., 90° F. or less, 80° F. or less, 70° F. or less) at about 1 atm of pressure. For example, the plasma discharge 216 is an atmospheric cold plasma operated at atmospheric pressures and conditions.

Non-thermal plasmas can affect the surface energy of product 205 without visibly damaging the product 205. For example, such discharge can reduce overheating or other phenomena that visibly affect the surface of the product 205, and/or reduce a shelf-life of product 205.

For example, plasma apparatus 212 creates an electric potential of about 5 kV to about 200 kV (e.g., about 5 kV to about 200 kV, about 20 kV to about 200 kV, about 40 kV to about 200 kV, about 60 kV to about 200 kV, about 80 kV to about 200 kV, about 5 kV to about 70 kV, about 5 kV to about 70 kV, or about 5 kV to about 30 kV) to generate the plasma discharge 216 for treating a surface of a product 205. Higher electric potentials can produce higher electron density values and/or higher reactive species densities within the plasma discharge 216. This can correspond with reduced residence times, and increased product 205 surface charge densities.

The discharge electrode 214 is operated at a current of less than 1 A (e.g., less than 1 A, less than 0.8 A, less than 0.6 A, less than 0.4 A, or less than 0.2 A). Lower current values permit energy efficient operation of the plasma apparatus 212. The plasma apparatus 212 operates discharge electrode 214 as an electric anode (e.g., a positive electrode) or as an electric cathode (e.g., a negative electrode).

The discharge electrode 214 is composed of a conductive metal such as steel, tungsten, titanium, cobalt, or combinations thereof. The plasma apparatus 212 includes a discharge electrode 214 in the shape of a needle with a sharp tip 215. Alternatively or additionally, the discharge electrode 214 includes a wire, a blade, a surface with an edge, a wedge point, or combinations thereof. In various embodiments, one or more discharge electrodes 214 can be used e.g., two or more, such as a regular array of electrodes.

The high voltage induces an electric field at the tip 215 of the discharge electrode 214 of sufficient intensity to ionize gas molecules in proximity to the tip 215, generating a plasma of free electrons and ionized molecules (e.g., ions). The area of high electric field in which free electrons and reactive species are present is a plasma discharge 216 which extends from the tip 215. The distance extended can depend on the strength of the electric field and, in some embodiments, is in a range from 1 cm to 10 cm. In some embodiments, the plasma discharge 216 imparts a charge on the surface of the product 205 exposed to the plasma discharge 216.

The plasma treatment device 210 imparts a surface charge on the product 205 during exposure to the plasma discharge 216, increasing the product 205 surface charge density (σₛ). In some embodiments, the plasma treatment device 210 increases as by at least 0.01 μC/m² (e.g., at least 0.1 μC/m², at least 0.2 μC/m², at least 0.5 μC/m², at least 1 μC/m², or at least 5 μC/m²). Higher surface charge densities increase the surface energy of a product 205 and increase wettability.

The plasma treatment device 210 electric field generates ions within the plasma discharge 216. In some embodiments, the plasma treatment device 210 generates an ion density within the corona discharge 216 of about 0.01×10⁶ ions/cm³ to about 200×10⁶ ions/cm³ (e.g., about 0.1×10⁶ ions/cm³ to about 200×10⁶ ions/cm³, about 1×10⁶ ions/cm³ to about 200×10⁶ ions/cm³, about 10×10⁶ ions/cm³ to about 200×10⁶ ions/cm³, about 50×10⁶ ions/cm³ to about 200×10⁶ ions/cm³, about 100×10⁶ ions/cm³ to about 200×10⁶ ions/cm³, about 150×10⁶ ions/cm³ to about 200×10⁶ ions/cm³, about 0.01×10⁶ ions/cm³ to about 150×10⁶ ions/cm³, about 0.01×10⁶ ions/cm³ to about 100×10⁶ ions/cm³, about 0.01×10⁶ ions/cm³ to about 50×10⁶ ions/cm³, about 0.01×10⁶ ions/cm³ to about 10×10⁶ ions/cm³, about 0.01×10⁶ ions/cm³ to about 1×10⁶ ions/cm³, or about 0.01×10⁶ ions/cm³ to about 0.1×10⁶ ions/cm³). Higher ion densities increase charge flux and reactive species (described below) present in the plasma discharge 216 increasing the rate of σₛ generation and increasing the rate of biological marker reduction.

The plasma discharge 216 includes a power density at the transport surface 202 within the treatment area 218. In some embodiments, higher treatment area 218 power densities can reduce the product 205 residence time. For example, the plasma discharge 216 power density can be 1 mW/s/mm² (1 mJ/mm²) or more (e.g., 10 mW/s/mm² or more, 100 mW/s/mm² or more, or 1000 mW/s/mm² or more).

The plasma treatment device 210 generates reactive species in the plasma discharge 216 such as reactive oxygen species (ROS), or reactive nitrogen species (RNS). The reactive species interact with the product 205 surface and affect the surface chemistry. The ROS present in the plasma discharge 216 can react with molecular bonds on the product 205 surface and change the bond composition. For example, untreated product 205 surfaces can include a high proportion of C—C bonds between molecular groups. Exposing the product 205 surface to the ROS present in the plasma discharge 216 alters the composition of molecular bonds by increasing the oxygen-containing bond structures. As specific examples, exposing the product 205 surface to ROS within the plasma discharge 216 can increase the proportion of O—C═O, C═O, N—C═O, C—O, and/or C—O—C bonds present on the product 205 surface. Increasing the proportion of these compounds increases the proportion of hydrophilic bond groups and increases the hydrophilicity of the surface.

In some embodiments, the reactive species affect the microbial load present on the product 205 surface. In some embodiments, exposing the product 205 to the plasma discharge 216 provides a surface disinfection by reducing the microbial load. ROS and RNS generated by CD, include assemblies of O₃, O₂, O., H₂O₂, NO, NO₂, HNO₃, HNO₂, ONOO⁻ and OH. which cause oxidative damage in molecular targets leading to DNA, protein, and lipid breakdowns leading to cell death.

Electric field, ion density, and reactive species (RNS and ROS) increase with increased electrode 214 voltage, and contribute to a plasma treatment device 210 disinfection effect (e.g., reduction of microbial load). Determining the disinfection effect can include the determination of a microbial load log reduction. A microbial load log reduction is the reduction of detectable microbial load markers (e.g., bacteria or viruses) by a logarithmic power value, for example, a log reduction of 1 corresponding to a reduction of detectable biologic markers by a factor of 10 (10⁻¹), or a log reduction of 2 corresponding to a reduction by a factor of 100 (10⁻²). In some embodiments, the log reduction can be in a range from 1 to 6 (e.g., 2 to 6, 3 to 6, 4 to 6, 5 to 6, 1 to 5, 1 to 4, 1 to 3, or 1 to 2). Higher log reductions reduce microbial load on product 205 surfaces and decrease the microbial load transfer between product 205 contact with the transport surface 202 or adjacent products 205.

The product 205 exposed to the plasma discharge 216 for a residence time is said to plasma-treated product 205. The residence time depends on, in an example embodiment, at least one of the treatment intensity, transportation speed of the transport surface 202, product 205 tumbling speeds, and product 205 surface area. The residence time for a plasma-treated product 205 can be in a range from 1 s to 300 s (e.g., 30 s to 300 s, 60 s to 200 s, 1 s to 60 s, or 1 s to 30 s). Increased residence time can increase product 205 σₛ, decrease the microbial load present on product 205 contact surfaces, increase product 205 exposure to ROS and RNS, and increase product 205 surface energy. Low residence times can increase product 205 mass rate through the coating system 100.

The product 205 can be products such as apples, citrus, berries, melons, peppers, tomatoes, leafy produce, fruits, vegetables, legumes, nuts, flowers, processed food products, candy, vitamins, nutritional supplements, an apple, asparagus, an apricot, an avocado, a banana, a blueberry, a bayberry, a cherry, a clementine mandarin, a cucumber, a custard apple, a fig, a grape, a grapefruit, a guava, a kiwifruit, a lime, a lychee, a mamey sapote, a mango, a melon, a mountain papaya, a nectarine, an orange, a papaya, a peach, a pear, a pepper, a persimmon, a pineapple, a plum, a strawberry, a tomato, a watermelon, and the like, and/or combinations thereof.

In some alternative embodiments, the plasma treatment device 210 generates the electric field within the plasma apparatus 212 and generates and emits a plasma discharge (e.g., a plasma plume), such as plasma discharge 216, using a process gas. Optionally, the emitted plasma discharge can be directed and/or shaped through a directing structure, such as through one or more nozzles, onto the transport surface 202 and/or product 205 surface(s). The plasma apparatus 212 is configured such that an internal discharge electrode, such as discharge electrode 214, is arranged within the plasma apparatus 212. The plasma apparatus 212 applies the voltage to the internal discharge electrode and generates the electric field. The electric field creates a plasma of reactive species, ions, and free electrons.

The plasma apparatus 212 flows a process gas around the internal discharge electrode. The process gas can be a homogenous molecular gas, or heterogeneous blend of gases. In some examples, the process gas can be a noble gas (e.g., Argon or Xenon), an atomic gas (e.g., Helium or Nitrogen), a molecular gas (e.g., O₂, N₂, NO, or NO₂), or a gas mixture (e.g., heloix, air, or other combinations of atomic and/or molecular gases). The process gas can be supplied to the plasma apparatus 212 via an external storage medium (e.g., a tank), an external source (e.g., a supply gas line), or the process gas can be received from the surrounding environment (e.g., air) and directed between the internal discharge electrode.

Process gas introduced to the internal discharge electrode is ionized and provides a source for the creation of reactive species. The bulk process gas flows around the internal discharge electrode and carries the generated ions, electrons, and reactive species through the directing structure. The process gas flowing through the one or more directing structures generates a plasma discharge, such as the plasma discharge 216 of FIG. 2B. The directing structures from which the plasma discharge emits can be oriented above the transport surface 202, or product 205 surface, in similar distances ranges as distances, D and d.

FIG. 2B is a top-down view of the product 205 within the treatment area 218 of plasma discharge 216. The treatment area 218 is area beneath plasma apparatus 212 in which the plasma discharge 216 contacts a surface, such as the transport surface 202 of FIG. 2A. The treatment area 218 extends across a portion of the surface 202 and in some embodiments can extend across the entire surface 202 width. The treatment area 218 is generally larger than the product 205 such that the plasma discharge 216 is in contact with the product 205 for a time period (e.g., a residence time). The surface area of the product 205 in contact with the plasma discharge 216 can be considered a contact area and in general the treatment area 218 is larger than the contact area. For example, a discharge electrode 214 a distance, D, from the transport surface 202 creates a treatment area 218 10% or more greater than the product 205 contact area (e.g., 10% greater, 20% greater, 50% greater, or 90% greater). Larger treatment area 218 with respect to product 205 contact area increases the residence time of the product 205 within the plasma discharge 216 and increases treatment effectiveness.

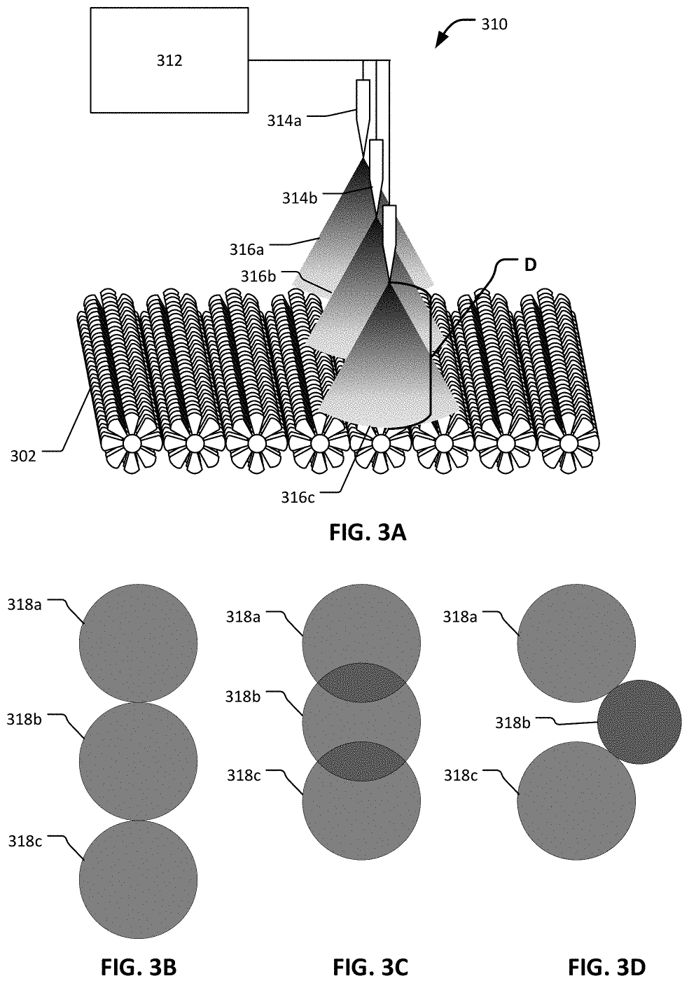

The shape, size, and treatment intensity of a treatment area, such as the treatment area 218 of FIG. 2B, can be achieved through combinations of discharge electrodes 214 of varying shapes, and distances from the surface, for example. FIG. 3A depicts a plasma apparatus 312 electrically connected to three needle-shaped discharge electrodes 314a, 314b, and 314c oriented above a brush bed conveyer 302. Each discharge electrode 314a, 314b, and 314c produces a respective plasma discharge 316a, 316b, and 316c which extends from the tip of each discharge electrode 314a, 314b, and 314c to the top surface of the conveyer 302.

In an example embodiment, the needle-shaped discharge electrodes produce a circular treatment area (e.g., on the surface of the conveyer 302), through which a product 205 passes while being transported on the conveyer 302. FIGS. 3B-3D schematically illustrate treatment areas 318a, 318b, and 318c of three needle-shaped discharge electrodes arranged in various example arrangements. The treatment areas 318a, 318b, and 318c are depicted cross sections of plasma discharges 316a, 316b, and 316c at the conveyer 302 surface. Each treatment area 318a, 318b, and 318 covers a portion of the conveyer 302 width and in some embodiments, can collectively cover the entire width.

FIG. 3B illustrates the treatment areas 318a, 318b, and 318c corresponding the discharge electrode 314a, 314b, and 314c arrangement of FIG. 3A. Each discharge electrode 314a, 314b, and 314c is spaced such that the center-to-center spacing of the treatment areas 318a, 318b, and 318c is equal to the discharge radius at the distance, D, between discharge electrodes 314a, 314b, and 314c and the conveyer 302. For example, each discharge electrode 314a, 314b, and 314c is spaced such that the center-to-center spacing of the treatment areas 318a, 318b, and 318c is within about 10% of the discharge radius at the distance, D, between discharge electrodes 314a, 314b, and 314c and the conveyer 302.

FIG. 3C illustrates treatment areas 318a, 318b, and 318c corresponding to an arrangement in which the discharge electrode 314a, 314b, and 314c are spaced such that the center-to-center spacing is less than the discharge radius at the distance, D. In such arrangements, areas of overlap between treatment areas 318a, 318b, and 318c can have higher treatment intensities than areas of non-overlap, as depicted by the darker shading of the overlapping areas. In some embodiments, higher treatment intensities can reduce treatment times to achieve similar increases in product 205 surface energies. In some embodiments, the energy intensity, frequency, or other parameter varies between discharge electrodes 314a, 314b, and 314c. Variation can facilitate consistent treatment parameters (e.g., across a width of the conveyor surface), such as by accounting for overlapping areas of adjacent discharge electrodes 314a, 314b, and 314c.

In various example embodiments, discharge electrodes are offset from one another in vertical and/or horizontal directions. For example, discharge electrodes 314a, 314b, and/or 314c can be offset from one another in vertical and/or horizontal directions. FIG. 3D illustrates the treatment area corresponding to an arrangement in which discharge electrodes 314a and 314c are aligned such that the plasma discharges 316a and 316c are not in contact. For example, electrodes 314a and 314c are spaced from one another (e.g., horizontally) a distance greater than a diameter of their respective outputs at conveyer 302 surface. Alternatively or additionally, the electrodes 314a and 314c are arranged at a same height relative to conveyer 302 surface (e.g., distance D is the same for each of electrodes 314a and 314c. The discharge electrode 314b is not aligned with discharge electrodes 314a and 314c. For example, electrode 314d is at a lower height such that a distance D between electrode 314d and the conveyer 302 surface is less than a distance D of electrodes 314a and 314c. Alternatively or additionally, the discharge electrode 314d is at a different position along conveyer 302 (e.g., at a relatively more advanced position) relative to discharge electrodes 314a and/or 314c. The lower distance decreases the plasma discharges 316b radius and/or increases the treatment intensity. In an example embodiment, the plasma discharge 316b circumference tangentially intersects the circumference of plasma discharges 316a and 316c.

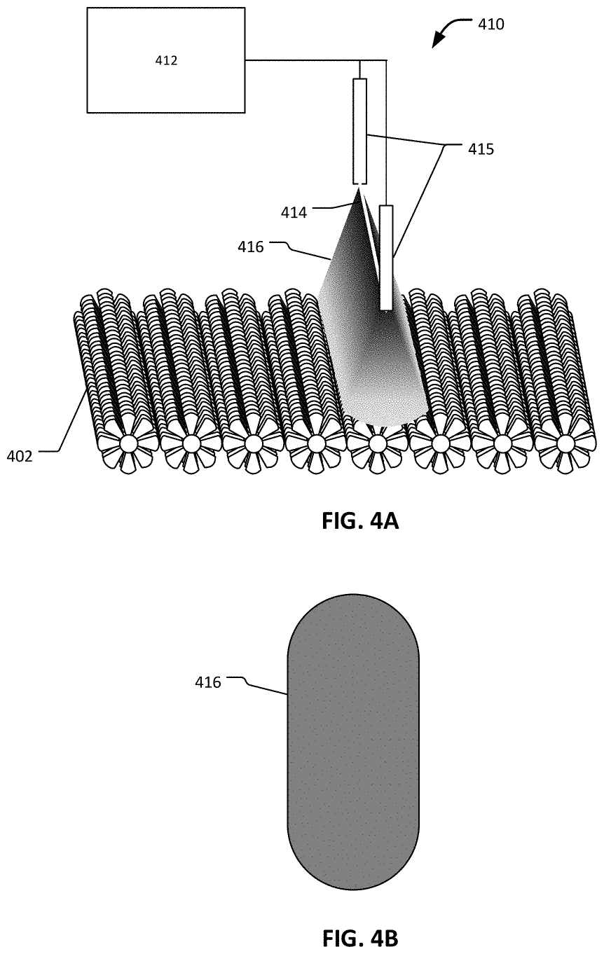

As described above, in some embodiments, a coating device 220 includes a discharge electrode 214 which produces a non-circular plasma discharge 216. FIG. 4A shows an example plasma treatment device 410 that includes plasma apparatus 412 electrically connected to a wire-shaped discharge electrode 414. The wire-shaped discharge electrode 414 extends between two electrical contacts 415. In an example embodiment, the wire-shaped discharge electrode 414 extends parallel with the conveyer 402 surface and perpendicular to the conveyer 402 path of travel. The plasma discharge 416 is approximately (e.g., substantially) symmetric along the length of the discharge electrode 414 and extended to the conveyer 402. The plasma discharge 416 of a wire-shaped discharge electrode 414 provides a consistent treatment area that extends the width of the conveyer 402 facilitating consistent treatment of products 205 traveling along conveyer 402.

FIG. 4B illustrates the treatment area corresponding to the wire-shaped discharge electrode 414 arrangement of FIG. 4A in which the treatment area longitudinal axis of symmetry is parallel with the discharge electrode 414.

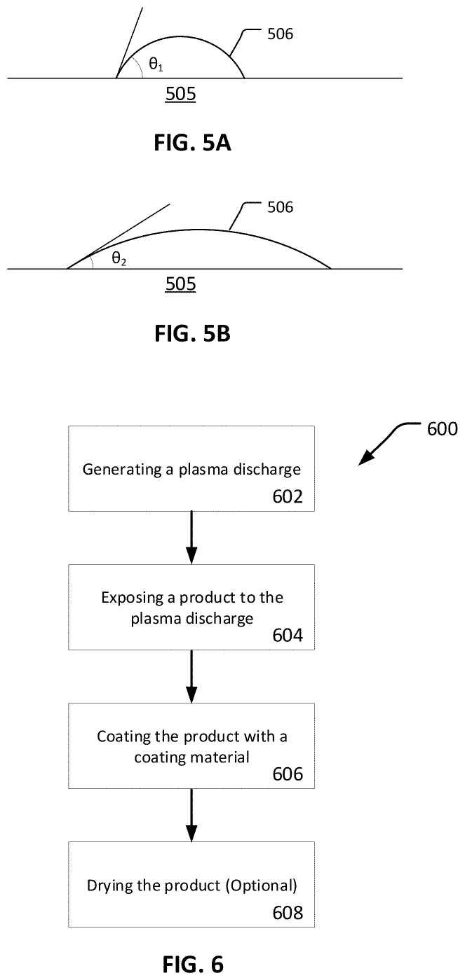

A product 205 traversing a treatment area undergoes a surface treatment from the plasma discharge 216 in which the molecules of the product 205 surface or the product 205 surface charge can be modified. The treated product 205 surface can have higher surface wettability when compared to untreated product 205 surfaces. FIGS. 5A and 5B compare cross-sections of a liquid-solid interface with a surface 505 having lower (5A) and higher (5B) surface wettability.

The contact angle (θ) the edge of a liquid makes compared to a solid surface provides a measure of the wettability of a surface. The contact angle is determined by the balance between adhesive forces, e.g., forces between molecules of different types, and cohesive forces, e.g., forces between molecules of the same type. In terms of a liquid droplet on a solid surface, the adhesive forces present in the contact interface adhere the liquid to the surface causing the liquid to spread, decreasing θ. The cohesive forces of the liquid attempt to bring the liquid surface to the lowest energy state, e.g., the smallest surface area, causing the liquid surface to contract, increasing θ. The balance of these forces results in an interface between the liquid and solid defined at least in part by θ, providing an inverse measure of surface wettability.

Molecules within the body or bulk of a material are surrounded in all directions by other molecules balancing the internal chemical and electrostatic potentials within the material. At a material surface, surface molecules are not surrounded leaving unbalanced chemical and electrostatic potentials, the sum of which can be called the ‘surface energy’ of a material surface. Plasma treatments remove hydrogen from the surface and attaching oxygen-containing species and other functional groups to the surface, increasing the surface energy of the material.

When a liquid comes into contact with a surface, the liquid material can spread which allows some of the liquid material to balance the solid material surface energy by spreading and forming the contact angle θ described above. Higher solid material surface energy causes higher adhesive forces resulting in higher liquid material spreading and in a lower contact angle θ.

As seen in FIGS. 5A and 5B, a liquid droplet 506 is in contact with a product surface 505 forming contact angle θ₁ which depends on at least the product surface 505 surface energy. The liquid droplet 506 can be any liquid including pure liquids, mixtures, or suspensions.

FIG. 5B depicts the liquid droplet 506 in contact with the product surface 505 following a plasma treatment in a plasma treatment device 210, such as any of the plasma treatment devices 210 described herein (e.g., 310, 410, etc.). The plasma treatment causes an increase in the surface energy of a product surface 505, and a decrease in the contact angle in which liquid droplet 506 and product surface 505 create a second contact angle θ₂. Because θ₂<θ₁, the liquid droplet 506 material spreads over a higher product surface 505 area creating a higher contact area per liquid material (e.g., spreading efficiency) than in FIG. 5A and depicting a product surface 505 with higher wettability.

FIG. 6 is a flow chart diagram depicting an example method 600 of plasma treating and coating a product. Method 600 includes operation 602 of generating a plasma discharge. The plasma discharge can be generated by a plasma treatment device creating an electric potential at a discharge electrode. In an example embodiment, operation 602 includes generating a plasma discharge that extends to an electrically neutral surface (e.g., conveyor). The discharge electrode shape, electric potential created and power supplied by the plasma apparatus, and gas composition surrounding the product and discharge electrode can be selected based on the shape, size, treatment intensity, treatment area, and chemistry of the plasma discharge, for example.

Surfaces of a product are exposed to the plasma discharge (step 604) for a treatment time. The treatment time can be controlled to deliver a predetermined exposure of product. In an example embodiment, determined by the product speed through the treatment area. Exposing the product surfaces to the plasma discharge removes hydrogen and attaches oxygen-containing species to the product surface that increases the surface energy. Increasing the surface energy of the product increases the wettability of the product surface when brought into contact with a liquid material.

A liquid coating material is applied to the product that coats the product surfaces (step 606) exposed to the liquid coating material. The increased wettability of the product surfaces facilitates efficient coating by decreasing the liquid-solid contact angle θ and increasing liquid material spreading.

Optionally, the liquid coating material is dried (step 608) to create a coating on the product 205. Drying solvent from the liquid coating material creates a stable coating that increases durability and shelf stability and decreases water mass loss during product transport and storage.

### EXAMPLES

### Example 1

### Cherry Tomato Treatment Groups

Four treatment groups of forty cherry tomatoes (n=40) were weighed to determine an initial group mass. Each treatment group was exposed to unique treatment conditions: one group was left untreated, one group plasma treated, one group coated in a liquid coating material, and one group plasma treated and coated in a liquid coating material and the mass loss factor was determined after a 12 day period for each group.

Groups that were plasma treated were exposed to a plasma discharge produced by a non-thermal plasma device, e.g., such as a Piezobrush PZ3 produced by TDK Electronics, arranged approximately 15 mm from a cherry tomato surface. Each cherry tomato was exposed to the plasma discharge by exposing a first surface to the plasma discharge at 100% power (8.0 W) for 60 s and then rotating the cherry tomato such that a second surface opposite the first surface is exposed to the plasma discharge for 60 s.

Groups that were coated in a liquid coating material were submerged in a vessel containing a volume of liquid coating material including a mixture of a monoglyceride and a fatty acid salt (including 94% w/v glycerol monostearate and 6% w/v sodium stearate) in a solvent (e.g., water) mixed to a final concentration of 40 g/L. The groups were agitated, removed, maintained on an air-exposed surface for 24 hours and the mass of each group determined. Maintaining and weighing the groups of cherry tomatoes was repeated for a total of 12 days and a ‘mass loss rate’ and ‘mass loss factor’ was determined for each group.

The term “mass loss factor” refers to the ratio of the average mass loss rate of uncoated plant matter (measured for a control group) to the average mass loss rate of the corresponding tested plant matter (e.g., coated plant matter) over a given time. The term “mass loss rate” refers to the rate at which the product loses mass (e.g. by releasing water and other volatile compounds). The mass loss rate is typically expressed as a percentage of the original mass per unit time (e.g. percent per day). Hence a larger mass loss factor for a coated plant matter corresponds to a greater reduction in average mass loss rate for the coated plant matter.

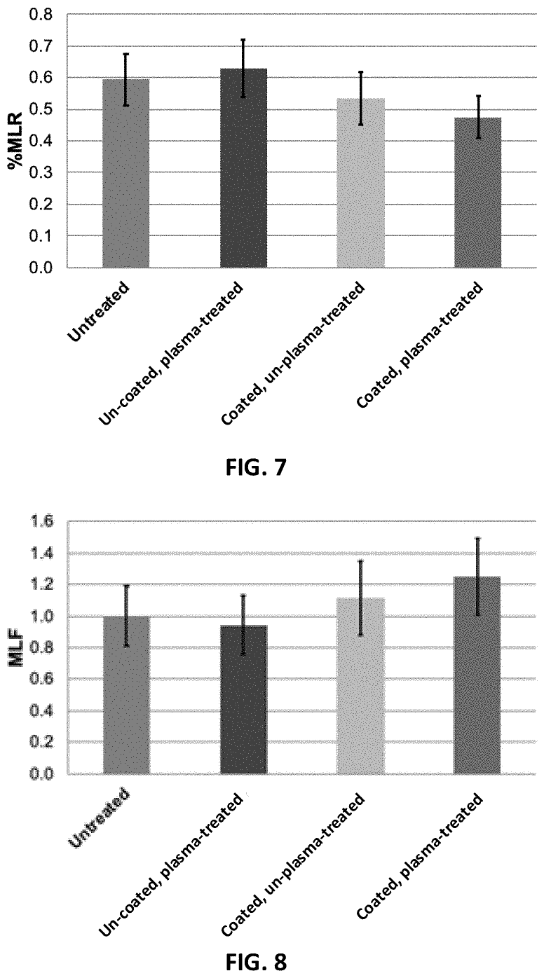

FIG. 7 is a bar chart comparing mass loss rate (% MLR) on the y-axis in a series of experimental conditions denoted on the x-axis. The vertical height of each column corresponding to a cherry tomato treatment group listed on the y-axis depicts the average % MLR determined at the end of the trial period. The treatment group average % MLR for each column is calculated by averaging the % MLR across all sub-groups, and the associated error bars is calculated as the % MLR standard deviation across all sub-groups.

The average % MLR for the untreated group (first, left-most column) was 0.59, the average % MLR for the un-coated, plasma-treated group (second column from the left) was 0.63, the average % MLR for the coated, un-plasma-treated group (third column from the left) was 0.53, and the average % MLR for the coated, plasma-treated group (fourth column from the left) was 0.47, the lowest of the compared groups.

FIG. 8 is a bar chart comparing mass loss factor (MLF) on the y-axis in a series of experimental conditions denoted on the x-axis. The vertical height of each column corresponding to a group of cherry tomatoes listed on the y-axis depicts the mass loss factor determined at the end of the trial period. The treatment group average MLF for each column is calculated by averaging the MLF across all sub-groups, and the associated error bars is calculated as the MLF standard deviation across all sub-groups.

The MLF for the untreated group (first, left-most column) was used as a normalization factor for the MLF of all groups, and therefore has a MLF of 1.0. The un-coated, plasma-treated group (second column from the left) had a final average MLF of 0.94, the coated, un-plasma-treated group (third column from the left) had a final average MLF of 1.11, and the coated, plasma-treated group (fourth column from the left) had the highest final average MLF of 1.25.

The mass lost during the 12 day trial period is predominantly water mass, e.g., dehydration, or desiccation. Reductions in water mass cause reductions in total cherry tomato volume which are visually quantifiable The sub-groups of cherry tomatoes for each treatment group were monitored for reduction in circumference (e.g., shrinkage). Images were taken of each sub-group of cherry tomatoes within the treatment groups at day 1, day 5, day 10, and day 12 of the trial period. The circumference and diameter of each tomato in each sub-group was determined using software-based measuring tools. The amount of circumferential reduction (e.g., % shrink) was determined for each sub-group using:

$$\% shrink = 1 - \frac{{circumference}_{day 5}}{{circumference}_{day 0}}$$

and the sub-group results averaged to determine the group average and standard deviation.

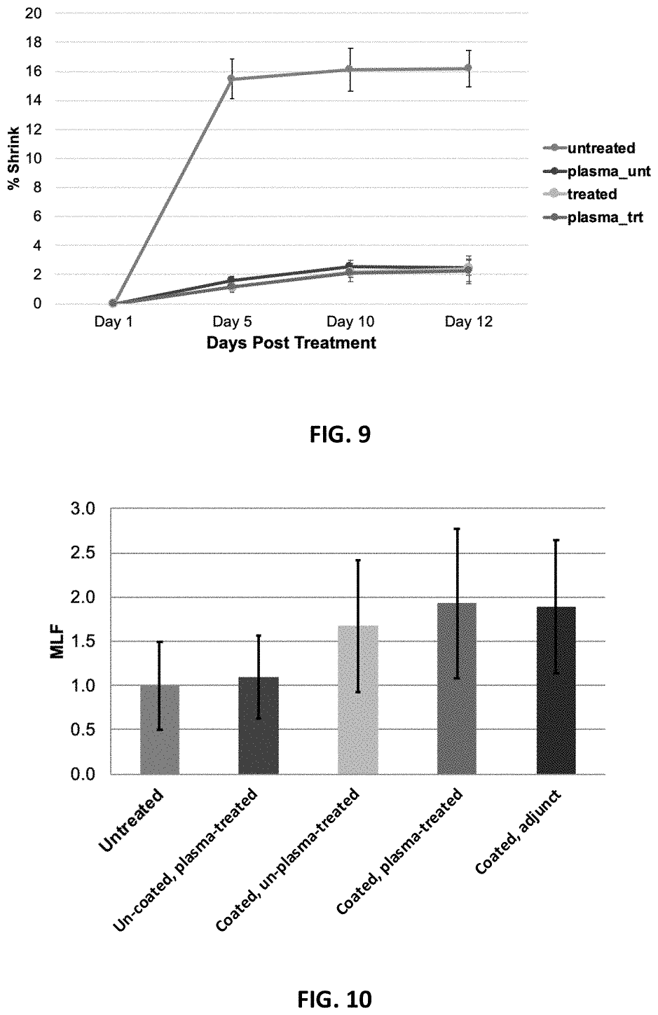

FIG. 9 is a line graph comparing circumferential reduction (e.g., % shrink) on the y-axis and time (in days) on the x-axis with time points at day 1, day 5, day 10, and day 12. The largest change in circumferential reduction is shown between day 1 and day 5 in all treatment groups with smaller changes in circumferential reduction between days 5, 10, and 12. The untreated group had a final circumferential reduction of about 16.2%, and the un-coated, plasma-treated group, coated, un-plasma-treated group, and coated, plasma-treated group had final circumferential reductions of about 2.3%.

### Example 2

### Kumquat Treatment Groups

A second example product was used to determine the efficacy of plasma treatment and coating in which five treatment groups of thirty kumquats (n=40) were divided into sub-groups of four kumquats and weighed to determine an initial group mass. The five treatment groups included untreated; un-coated, and plasma-treated; coated, and un-plasma-treated; coated, and plasma-treated; and coated including an adjunct.

The plasma treatment parameters and coating methods were the same for the kumquat treatment groups as described above for the cherry tomato treatment groups. For the coated including an adjunct kumquat treatment group, xanthan gum was added to the coating mixture to a final concentration of 0.2% w/v. As described above, a ‘mass loss rate’ and ‘mass loss factor’ was determined for each kumquat treatment group after 12 day trail period.

FIG. 10 is a bar chart comparing mass loss factor (MLF) on the y-axis in a series of experimental conditions denoted on the x-axis. The vertical height of each column corresponding to a kumquat treatment group listed on the y-axis depicts the mass loss factor determined at the end of the 12 day trial period. The treatment group average MLF for each column is calculated by averaging the MLF across all sub-groups, and the associated error bars is calculated as the MLF standard deviation across all sub-groups.

The MLF for the untreated group (first, left-most column) was used as a normalization factor for the MLF of all groups, and therefore has a MLF of 1.0. The un-coated, plasma-treated group (second column from the left) had a final average MLF of 1.10, the coated, un-plasma-treated group (third column from the left) had a final average MLF of 1.67, the coated, plasma-treated group (fourth column from the left) had the highest final average MLF of 1.93, and the coated including an adjunct group (fifth column from the left) had a final average MLF of 1.89.

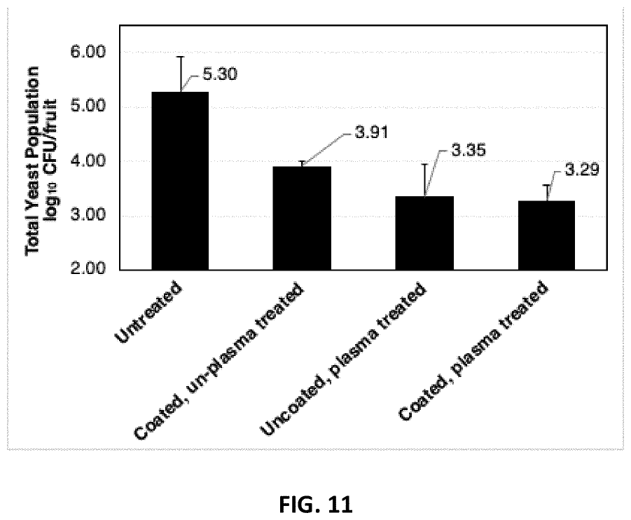

Referring now to FIG. 11, an example product (e.g., kumquat) was used to determine the efficacy of combined plasma treatment and coating in which four treatment groups of five kumquat replicates each (n=20) were analyzed to measure the natural background microflora, such as microflora existing on the surface of the treatment groups prior to coating and treatment, in terms of total generic yeast counts assessed in the form of colony forming units (CFU). The four treatment groups included untreated; un-coated, and plasma-treated; coated, and un-plasma-treated; and coated, and plasma-treated. The plasma treatment parameters and coating methods were the same for the kumquat treatment groups as described above for the cherry tomato treatment groups.

FIG. 11 is a bar chart comparing yeast populations recovered from kumquats on the y-axis in a series of experimental conditions denoted on the x-axis. The vertical height of each column corresponds to a kumquat treatment group listed on the x-axis depicts the total yeast counts measured in log₁₀ CFU/fruit after 4 hours following the experimental treatments. The treatment group average, displayed above and offset to the right from each respective column, total yeast count for each column is calculated by averaging the recovered counts of 5 fruits, and the associated error bars is calculated as the yeast count standard deviation across one group.

The average yeast count of untreated fruits equal to 5.30 log₁₀ CFU/fruit was used as the reference starting population. The coated un-plasma treated group showed had a survivor (e.g., remaining recoverable generic yeast) population of 3.91 log₁₀ CFU/fruit. The uncoated plasma treated fruit had a yeast population of 3.35 log₁₀ CFU/fruit, which was a 1.95 log₁₀ CFU/fruit reduction or approximately 98% reduction. The coated and plasma treated fruit had a yeast population of 3.29 log₁₀ CFU/fruit, with greater than 2 log reduction or greater than 99% reduction in the initial yeast counts. These results indicate that plasma treatment, and plasma treatment in combination with coating material application, is beneficial in decreasing generic yeast survivor populations on the surface of treated products.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A plasma coating system, comprising:
a plasma treatment device configured to generate a plasma discharge to treat a product;
a coating station that treats the product with a liquid coating material; and
a transport surface that transports a product from the plasma treatment device to the coating station.

2. The system of claim 1, wherein the plasma treatment device is arranged above the transport surface.

3. The system of claim 1, further comprising a drying station that receives products from the coating station and facilitates drying of the liquid coating to form a dried coating layer on the product.

4. The system of claim 3, wherein the plasma coating system comprises a second plasma treatment device configured to generate a second plasma discharge arranged between the coating station and the drying station.

5. The system of claim 1, wherein the coating station includes an applicator selected from the group consisting of a spray applicator, a brush-bed applicator, and a submersion applicator.

6. The system of claim 1, wherein the plasma treatment device is configured to generate the plasma discharge based on a peak electric potential in a range from 1 V to 200 kV.

7. The system of claim 1, wherein the plasma discharge is a non-thermal plasma discharge with a bulk temperature below 200° F.

8. The system of claim 1, wherein the plasma discharge covers an area in a range from 0.01 m² to 10 m².

9. The system of claim 1, wherein a minimum distance between the plasma treatment device and the product is in a range from 5 cm to 15 cm.

10. The system of claim 1, wherein the plasma discharge covers a surface dimension of the transport surface in a range from 0.1 m to 10 m.

11. The system of claim 1, wherein the transport surface is configured to transport the product at a rate such that the product is within the plasma discharge for a residence time, wherein the residence time is in a range from 1 s to 300 s.

12. The system of claim 1, wherein the liquid coating material comprises a monoglyceride and fatty acid salt, wherein the fatty acid salt comprises a C16 fatty acid salt and a C18 fatty acid salt.

13. A method for plasma treating and coating product, comprising:
generating a plasma discharge;
exposing an external surface of an product to the plasma discharge; and
applying a liquid coating material to the external surface of the product.

14. The method of claim 13, wherein exposing the product to the plasma discharge includes exposing the product to a non-thermal plasma discharge with a bulk temperature below 200° F. for a time duration in a range from 1 s to 10 m.

15. The method of claim 13, comprising, after applying the liquid coating material, exposing the product to a second plasma discharge.

16. The method of claim 13, wherein the liquid coating material comprises a monoglyceride and fatty acid salt, wherein the fatty acid salt comprises a C16 fatty acid salt and a C18 fatty acid salt.

17. A plasma-treated agricultural product, comprising:
an agricultural product having a plasma-treated surface that has been exposed to a plasma discharge, the plasma-treated surface having an elevated wettability.

18. The product of claim 17, comprising a liquid coating that covers at least a portion of the plasma-treated surface, wherein the coating comprises a monoglyceride and fatty acid salt, wherein the fatty acid salt comprises a C16 fatty acid salt and a C18 fatty acid salt.

19. The product of claim 18, wherein the liquid coating on the surface has a lowered contact angle that is more than 1° less than a coating contact angle on the product prior to being exposed to the plasma discharge.

20. The product of claim 17, wherein the plasma-treated surface has an increased hydrophilicity.
